SEQUENCE LISTING (9 sequences)

SEQ ID NO: 1 - DNA, 48 bp, Artificial sequence
  Synthetic nucleic acid sequence
ctatacgactctaggcgtgtaccatgtggttgtaaaacgacggccagt

SEQ ID NO: 2 - DNA, 50 bp, Artificial sequence
  Synthetic nucleic acid sequence
catgctacgtatcgcagtgattgcacaacgaacccactctgcttctgtgg

SEQ ID NO: 3 - DNA, 50 bp, Artificial sequence
  Synthetic nucleic acid sequence
ctatacgactctaggcgtgtaccatgtggtagtctcccaggtaccccact

SEQ ID NO: 4 - DNA, 51 bp, Artificial sequence
  Synthetic nucleic acid sequence
catgctacgtatcgcagtgattgcacaacgcaggaaacagctatgaccatg

SEQ ID NO: 5 - DNA, 51 bp, Artificial sequence
  Synthetic nucleic acid sequence
catgctacgtatcgcagtgattgcacaacgcgcaaatgggcggtaggcgtg

SEQ ID NO: 6 - DNA, 48 bp, Artificial sequence
  Synthetic nucleic acid sequence
ctatacgactctaggcgtgtaccatgtggtgatgcggcactcgatctc

SEQ ID NO: 7 - DNA, 20 bp, Artificial sequence
  Synthetic nucleic acid sequence
atcgcagtgattgcacaacg

SEQ ID NO: 8 - DNA, 20 bp, Artificial sequence
  Synthetic nucleic acid sequence
ctatacgactctaggcgtgt

SEQ ID NO: 9 - DNA, 20 bp, Artificial sequence
  Synthetic nucleic acid sequence
ctaggcgtgtaccatgtggt

(19) United States
(12) Patent Application Publication
(10) Pub. No.: US 2017/0130258 A1
(43) Pub. Date: May 11, 2017

(54) MULTIPLEX ON-ARRAY DROPLET PCR AND QUANTITATIVE PCR

(71) Applicant: Agilent Technologies, Inc., Santa Clara, CA (US)

(72) Inventor: Nicholas M. Sampas, San Jose, CA (US)

(21) Appl. No.: 15/261,469

(22) Filed: Sep. 9, 2016

Related U.S. Application Data
(60) Provisional application No. 62/253,546, filed on Nov. 10, 2015.

(51) Int. Cl. C12Q 1/68

(52) U.S. Cl. CPC C12Q 1/686; C12Q 1/6874

(57) ABSTRACT

This present disclosure provides methods and compositions for multiplex amplification, e.g., PCR, of DNA samples compartmentalized on the surface of an array. In certain aspects, the method includes providing a solid substrate having features of immobilized forward and reverse primer pair constructs therein for amplifying a target polynucleotide sequence, contacting the plurality of features with a corresponding nucleic acid sample, and incubating the contacted features under conditions to allow release of the forward and reverse primer constructs and amplification of the desire target polynucleotide. The release of the primer constructs from the substrate can be done prior to or during the incubating step.

### CROSS-REFERENCING

This application claims the benefit of provisional application Ser. No. 62/253,546, filed on Nov. 10, 2015, which application is incorporated by reference herein.

### FIELD OF THE INVENTION

The present invention relates to the field of molecular biology. More specifically, the invention relates to methods and compositions for multiplex amplification of polynucleotides on the surface of a solid substrate.

### BACKGROUND

Given the ever increasing capabilities of NGS technologies to sequence and deconvolute thousands of different barcoded DNA samples mixed in the same sequencing reaction, there is a need in the art to provide improved methods for sample barcoding and targeted amplification multiple individual DNA samples. The present disclosure provides methods and compositions that find use in this, and other, applications.

### SUMMARY

This disclosure describes methods and compositions that find use in highly multiplex PCR of nucleic acids in one or more samples compartmentalized on the surface of an array of features containing PCR primer pairs for target nucleic acids. The methods and compositions disclosed can be considered multiplex in both the number of samples analyzed and the number of target loci amplified from each sample. Multiplex PCR is useful for targeted sequencing, for example, of a panel of genes from a nucleic acid sample, e.g., genomic DNA sample. Such gene panels have applications to disease diagnostics and cancer genomics in both research and clinical markets. The disclosed methods and compositions can also be applied to multiplex single-cell sequencing of gene panels. In some embodiments, the disclosed methods and compositions employ reverse transcription PCR (rtPCR).

### BRIEF DESCRIPTION OF THE FIGURES

The skilled artisan will understand that the drawings, described below, are for illustration purposes only. The drawings are not intended to limit the scope of the present teachings in any way.

FIGS. 1A, 1B and 1C show diagrams of different embodiments of primer construct configurations according to aspects of the present disclosure.

FIG. 2 shows a diagram of two features with covalently attached oligonucleotides containing primer pair constructs linked by cleavable linkers.

FIG. 3 shows a diagram of two features with oligonucleotides containing primer pair constructs linked by cleavable linkers that are non-covalently attached to the solid substrate via hybridization of terminal linker sequences to capture oligonucleotides covalently attached to the substrate.

FIG. 4 shows a diagram of two features on an array that have been contacted with individual aqueous samples (droplets) containing target molecules. The aqueous droplets are encased in an immiscible fluid.

FIG. 5 shows a diagram of the sample-contacted features of FIG. 4 after release of the primer constructs via cleavage of the cleavable linkers.

FIG. 6 shows a diagram of two adjacent features on an array that have been contacted with a single aqueous sample (droplet) containing target molecules. The aqueous droplet is encased in an immiscible fluid.

FIG. 7 shows a diagram of an immobilization scheme for a reporter probe or molecular beacon that includes an indexing sequence in a feature with corresponding forward and reverse primers. This feature can be used to quantitatively report on one or more targets of interest.

FIG. 8 shows a diagram for amplification of a target sequence by nested PCR.

FIG. 9 shows a diagram of the immobilization scheme of the forward and reverse primer constructs employed in Example 1. In this embodiment, the forward and reverse primer constructs are immobilized via hybridization to the same capture oligonucleotide but at different locations. The capture/terminal linker sequences are referred to as “indexing sequences” in this figure.

FIG. 10 shows a diagram of the product of the first round of amplification of a target polynucleotide using the primer constructs shown in FIG. 9.

FIG. 11 shows a diagram of the second round of amplification of the product from the first round of amplification (FIG. 10) using primers specific for the indexing sequences at the termini of the product from the first round.

FIG. 12 shows BioAnalyzer chip data of products of amplification reactions performed in Example 1. Different lanes utilized different combinations of primers. Lanes 1-6 and 11 used all three primers, lanes 7 and 8 used primer pair 1, and lanes 9 & 10 used primer pair 2. All lanes showed all three amplicons for both the on-array droplet material and the positive control input material. Differences in product lengths between the use of primer pairs 1 and 2 can be seen in lanes 7-10.

### DEFINITIONS

Before describing exemplary embodiments in greater detail, the following definitions are set forth to illustrate and define the meaning and scope of the terms used in the description.

Numeric ranges are inclusive of the numbers defining the range. Unless otherwise indicated, nucleic acids are written left to right in 5′ to 3′ orientation; amino acid sequences are written left to right in amino to carboxy orientation, respectively.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

It must be noted that as used herein and in the appended claims, the singular forms “a”, “an”, and “the” include plural referents unless the context clearly dictates otherwise. For example, the term “a primer” refers to one or more primers, i.e., a single primer and multiple primers. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “array of features”, “microarray”, and the like are intended to mean a two-dimensional arrangement of addressable regions bearing particular moieties (e.g., biopolymers) associated with that region. Each different addressable region bearing one of more moieties is also called a “feature” (hence the term “array of features”). In some embodiments, an array is an array of polymeric binding agents, where the polymeric binding agents can be any of: peptides, oligonucleotides, polysaccharides, synthetic mimetics of such biopolymeric binding agents, etc. The oligonucleotides of an array can be covalently attached to substrate at any point along the nucleic acid chain, but are generally attached at one terminus (e.g. the 3′ or 5′ terminus). Sometimes, the arrays are arrays of polypeptides, e.g., proteins or fragments thereof.

Any given substrate can carry one, two, three, four or more arrays disposed on a front surface of the substrate. Depending upon the use, any or all of the arrays can be the same or different from one another and each can contain multiple spots (or features). An array can contain at least, inter alia, 10, at least 100, at least 1,000, at least 10,000, at least 100,000, or at least 10⁶ or more features, in an area of less than 20 cm², e.g., in an area of less than 10 cm², of less than 5 cm², or of less than 1 cm². In some embodiments, features can have widths (that is, diameter, for a round spot) in the range of, inter alia, from 1 μm to 1.0 cm, although features outside of these dimensions are envisioned. In some embodiments, a feature can have a width in the range of, inter alia, 3.0 μm to 200 μm, e.g., 5.0 μm to 100 μm or 10 μm to 50 μm. Interfeature areas will typically be present which do not carry any polymeric compound. It will be appreciated though, that the interfeature areas, when present, can be of various sizes and configurations.

Each array can cover an area of less than, inter alia, 100 cm², e.g., less than 50 cm², less than 10 cm² or less than 1 cm². In some embodiments, the substrate carrying the one or more arrays will be shaped generally as a rectangular or square solid (although other shapes are possible), having a length of, inter alia, more than 4 mm and less than 10 cm, e.g., more than 5 mm and less than 5 cm, and a width of more than 4 mm and less than 10 cm, e.g., more than 5 mm and less than 5 cm.

Arrays can be fabricated using drop deposition from pulse jets (or inkjets) of either polynucleotide precursor units (such as monomers) in the case of in situ fabrication, or the previously obtained polynucleotide. Such methods are described in detail in, for example, U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. These references are hereby incorporated by reference herein. Other drop deposition methods can be used for fabrication, as previously described herein. Also, instead of drop deposition methods, photolithographic array fabrication methods can be used. Interfeature areas need not be present particularly when the arrays are made by photolithographic methods.

An array is “addressable” when it has multiple regions of different moieties (e.g., different polynucleotide sequences) such that each region (i.e., a “feature”, “spot” or “area” of the array) is at a particular predetermined location (i.e., an “address”) on the array. Array features are typically, but need not be, separated by intervening spaces.

As used herein, the terms “solid substrate”, “solid support”, and the like, are used in accordance with their meaning in the art. They are thus any material known in the art as suitable for binding and retaining biomolecules, e.g., nucleic acids, under conditions of binding, purification and/or enzymatic reaction. Non-limiting examples of solid substrates useful in the present invention include: nylon, yttrium silicate (YSi), nitrocellulose, PVDF membranes, plastic surfaces (such as those comprising polystyrene or polypropylene), etc. Solid supports can be chemically modified, e.g., aminated (primary or secondary amine) or carboxylated to facilitate attachment of a particular moieties.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and can contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and can be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine, thymine, uracil (G, C, A, T and U respectively). DNA and RNA have a deoxyribose and ribose sugar backbone, respectively, whereas PNA's backbone is composed of repeating N-(2-aminoethyl)-glycine units linked by peptide bonds. In PNA various purine and pyrimidine bases are linked to the backbone by methylene carbonyl bonds. A locked nucleic acid (LNA), often referred to as an inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The term “unstructured nucleic acid”, or “UNA”, is a nucleic acid containing non-natural nucleotides that bind to each other with reduced stability. For example, an unstructured nucleic acid can contain a G′ residue and a C′ residue, where these residues correspond to non-naturally occurring forms, i.e., analogs, of G and C that base pair with each other with reduced stability, but retain an ability to base pair with naturally occurring C and G residues, respectively. Unstructured nucleic acid is described in US20050233340, which is hereby incorporated by reference herein for disclosure of UNA.

The term “oligonucleotide” as used herein denotes a single-stranded multimer of nucleotides of from about 2 to 500 nucleotides in length, e.g., from about 10 to 200 nucleotides. Oligonucleotides can be synthetic or can be made enzymatically, and, in some embodiments, are 30 to 150 nucleotides in length. Oligonucleotides can contain ribonucleotide monomers (i.e., can be oligoribonucleotides) and/or deoxyribonucleotide monomers. An oligonucleotide can be, inter alia, 10 to 20, 21 to 30, 31 to 40, 41 to 50, 51-60, 61 to 70, 71 to 80, 80 to 100, 100 to 150, 150 to 200, 200 to 300, 300 to 400, or 400 to 500 nucleotides in length, for example.

The term “primer” as used herein refers to an oligonucleotide, whether occurring naturally or produced synthetically, which is capable of acting as a point of initiation of nucleic acid synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer can be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it can contain fewer nucleotides. The primers herein are selected to be substantially complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment can be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

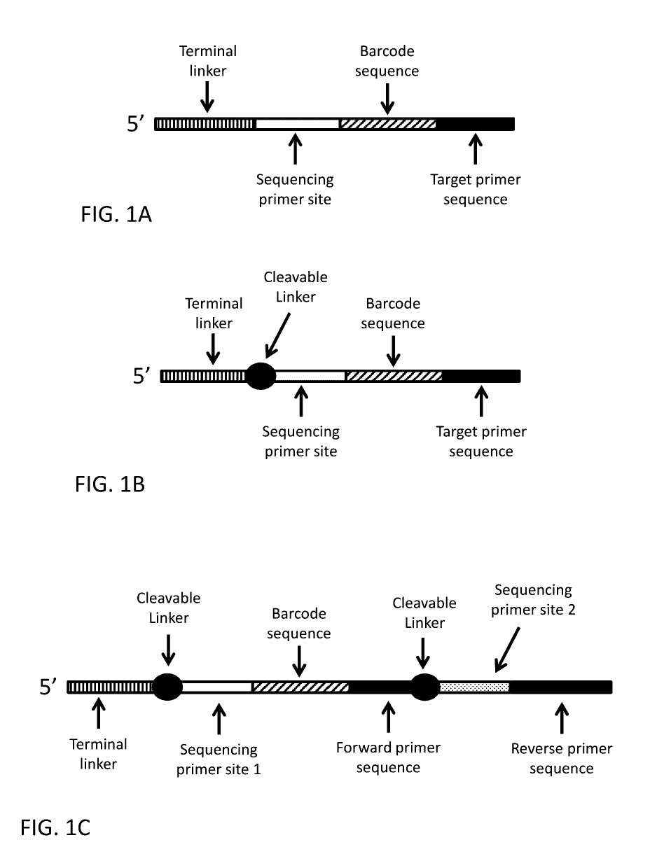

The term “primer construct” as used herein refers to a nucleotide sequence comprising a primer region and at least one other functional region, e.g., upstream of the primer region, e.g., a barcode sequence, a binding site for a universal sequencing primer, a terminal linker sequence/array indexing sequence, etc. (see, e.g., FIGS. 1A, 1B and 1C). The primer region of a primer construct is capable of acting as a point of initiation of nucleic acid synthesis when placed under conditions in which synthesis of a primer extension product, which is complementary to a nucleic acid strand, is induced (as described above for “primers”). The primer region is sometimes referred to as a target nucleic acid binding site.

The term “adapter” is used herein to refer to an oligonucleotide component, either double-stranded or single-stranded, that is ligated to a polynucleotide using any convenient method. In certain embodiments, an adapter is ligated to a DNA molecule that is being fragmented in a tagmentation reaction with a transposase complex.

As used herein, the term “peptide” is intended to refer to a polymer of amino acids or analogs thereof.

An array of polymeric compounds can be made using any suitable method, including methods in which pre-made polymeric compounds are deposited onto the surface of a substrate and then linked to the substrate, and also in situ synthesis methods.

As used herein, the term “synthesizing the polymeric compounds in situ” is intended to refer to methods by which a polymeric compound is grown in place on a substrate using monomeric precursors that are added one by one to a growing chain. Such methods include photolithographic methods, as well as drop deposition methods. Examples of such methods are described in, e.g., Cleary et al. (Nature Methods 2004 1: 241-248) and LeProust et al. (Nucleic Acids Research 2010 38: 2522-2540).

As used herein, the term “cleavable linker” is intended to refer to an arrangement in which a first component (e.g., a polymeric compound) is linked to a second component (e.g., a solid substrate or a second polymeric compound) via a cleavable bond. A cleavable bond can be cleaved using base (e.g., ammonia or trimethylamine), acid, fluoride, or photons, for example.

As used herein, the term “areas that contain the polymeric compounds on the surface of the substrate” is intended to refer to the features that contain the polymeric compounds, as discussed elsewhere herein.

As used herein, the term “remainder of the surface of the substrate” is intended to refer to the areas of the surface of the substrate that do not contain the polymeric compounds (i.e., the areas of the surface of the substrate that lie between the areas that contain the compounds).

As used herein, the terms “hydrophobic” and “hydrophilic” are relative terms and are intended to refer to the degree by which a solution is attracted to or repelled from a surface. Hydrophobicity and hydrophilicity can be measured by measuring the contact angle of the solution on the surface, as described in Johnson et al. (J. Phys. Chem. 1964 Contact Angle Hysteresis 68: 1744-1750). Contact angle is a measure of static hydrophobicity, and contact angle hysteresis and slide angle are dynamic measures. See also the paper entitled Contact Angle Measurements Using the Drop Shape Method by Roger P. Woodward, which can be obtained at the website formed by placing “http://www.” in front of “firsttenangstroms.com/pdfdocs/CAPaper.pdf”.

As used herein, the term “selectively hydrating” is intended to refer to a step in which an aqueous solution is selectively applied to a selected feature of an array of features (or to selected groups of features that are immediately adjacent to one another), but not to the areas in between the selected features/groups of features. This step results in a substrate that has an array of droplets on its surface, where the edges of the droplets correspond to the boundaries of the selected features/group of features.

As used herein, the term “discrete droplets” is intended to refer to droplets on the surface of a substrate that are separated from one another. As described elsewhere herein, each discrete droplet can occupy a single area (i.e., where each droplet lies over a single feature) or each discrete droplet can occupy multiple areas (where the droplets are actively induced to bleed into each other in a pre-defined way so that one droplet can contain multiple features).

As used herein, the term “each droplet contains a single compound” is intended to refer to a droplet that contains multiple molecules of the same substantially pure compound.

As used herein, the term “pre-defined” is intended to refer to something that is known prior to being made.

As used herein, the phrase “releasing a polymeric compound from a surface” or equivalent (e.g., “forward and reverse primer pair constructs are released from the substrate”) is intended to refer to incubating or treating one or more substrate-immobilized polymeric compounds in a manner such that they are freed from their immobilized state from the substrate (e.g., by denaturing from an indexing oligonucleotide on the substrate or cleaving a cleavable linker that holds the polymeric compound to the substrate).

As used herein, the term “collecting the droplets in an immiscible liquid” is intended to refer to a step in which droplets that are on the surface of a substrate are physically separated from the substrate to become droplets in an immiscible liquid, i.e., an emulsion.

As used herein, the term “emulsion” is intended to refer to a mixture of two or more liquids that are normally immiscible, in which one liquid forms droplets that are dispersed within another liquid. A water-in-oil emulsion refers to an emulsion that contains aqueous droplets and an organic (oily or hydrophobic) continuous phase. Depending on the liquids used, the droplets of an emulsion can be in the range of, inter alia, 100 nm to 100 μm, e.g., 1 μm to 50 μm.

As used herein, the term “droplet” is intended to refer to the aqueous part of an emulsion that is interspersed in a continuous liquid that is immiscible with water (i.e., the immiscible liquid).

As used herein, the term “immiscible liquid” or “immiscible fluid” is intended to refer to a continuous part of an emulsion.

As used herein, the term “in the solution phase” is intended to refer to a polymeric compound that is in an aqueous environment that is not bound or tethered to a solid substrate. Such a polymeric compound can be dissolved in the aqueous environment.

As used herein, the term “adjacent to one another on the substrate” is intended to refer to areas that contain polymeric compounds that are immediately adjacent to one another (i.e., next to each other, without any other areas that contain polymeric compounds that are in between).

As used herein, the term “mixture” is intended to refer to a solution in which the components are interspersed with one another and not spatially separated.

As used herein, the term “aqueous” is intended to refer to a medium in which the solvent is water.

As used herein, the term “a plurality of molecules of the compound(s)” is intended to refer to a composition that contains multiple molecules of the same compound. For example, a solution containing at least 100 molecules of a compound(s) contains at least 100 molecules of the same compound. More specifically, if a droplet contains at least 100 molecules of a particular oligonucleotide, then it contains at least 100 molecules of the same oligonucleotide.

A “plurality” contains at least 2 members. In certain cases, a plurality can have, inter alia, at least 10, at least 100, at least 1,000, at least 10,000, at least 100,000, at least 10⁶, at least 10⁷, at least 10⁸ or at least 10⁹ or more members.

Other definitions of terms can appear throughout the specification.

### DETAILED DESCRIPTION

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

All patents and publications, including all sequences disclosed within such patents and publications, referred to in the present disclosure are hereby expressly incorporated by reference herein.

Aspects of this disclosure are drawn to methods, compositions, and kits for multiplex amplification of target polynucleotides of one or more nucleic acid samples on the surface of a microarray.

Thus, aspects of the present disclosure provide a method that includes: (1) providing a solid substrate comprising a plurality of oligonucleotide features (e.g., a microarray) in which each of the plurality of oligonucleotide features includes a substrate-immobilized forward primer construct having a first target nucleic acid binding site and a substrate-immobilized reverse primer construct having a second target nucleic acid binding site, wherein the first and second target nucleic acid binding sites form a primer pair for amplifying a target polynucleotide sequence; (2) contacting each of the plurality of features with a corresponding nucleic acid sample; and (3) incubating the contacted plurality of features under conditions to allow amplification of the target polynucleotide sequence by the forward and reverse primer pair constructs. Either prior to or during the incubating step, the forward and reverse primer pair constructs are released from the substrate.

Amplification of a target polynucleotide sequence by PCR requires a pair of primer oligonucleotides (or primer constructs) generally referred to in the art as forward and reverse primer pairs (see above). Forward and reverse primers are designed to hybridize to opposite strands of a target nucleic acid sequence and prime nucleic acid synthesis toward each other. Typically, the region of a primer (or primer construct) that is complementary to the priming site on a target nucleic acid (the target nucleic acid binding site) ranges from about 15 to about 35 nucleotides, e.g., from about 18 to about 25 nucleotides. The design of primer pair sequences for a target nucleic acid sequence is within the skill of the art.

In addition to the region complementary to the priming site on a target nucleic acid, a primer (or primer construct) can include at least one other region or domain (e.g., as shown in FIGS. 1A, 1B and 1C), which can contain specific functional sequences, including terminal linker sequences, barcode sequences, primer binding sites for amplification or other polymerization reactions (e.g., sequencing), restriction enzyme recognition sites, etc. Certain of these regions/domains are described in further detail below. In some cases, a single domain is multi-functional, e.g., a terminal linker sequence can also be a primer binding site for a next generation sequencing primer.

As indicated above, the forward and reverse primer pair constructs are immobilized to the solid support (the array). In certain embodiments, the forward and reverse primer pair constructs are immobilized via hybridization of a terminal linker sequence to a complementary sequence on a capture oligonucleotide covalently attached to the solid substrate (see FIG. 3). Thus, the terminal linker sequence and the capture oligonucleotide are complementary to one another. In certain embodiments, the terminal linker sequence is present at the 5′ end of the primer construct (a 5′ terminal linker) whereas in other embodiments the terminal linker sequence is present on the 3′ end of the primer construct (a 3′ terminal linker). In some instances, the terminal linker is cleavably linked to the target nucleic acid binding site such that it is released prior to or during the incubating step (e.g., during amplification). While many of the embodiments below are described using a 5′ terminal linker sequence, this is not meant to exclude the use of a 3′ terminal linker sequence where appropriate.

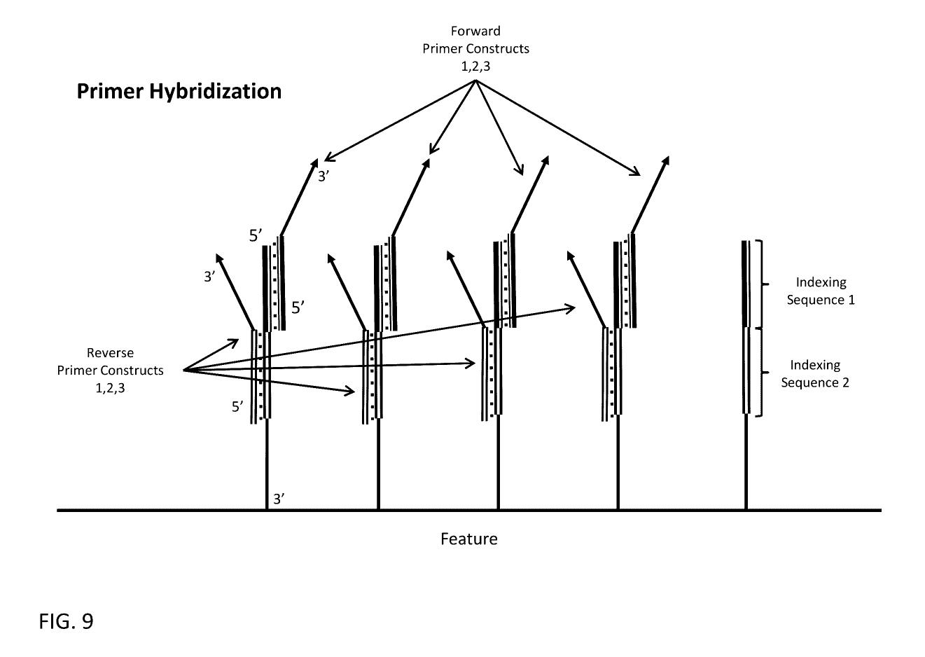

In certain embodiments, the forward and reverse primer pair constructs each comprise the same 5′ terminal linker sequence. In certain implementations of this embodiment, a mixture of the forward and reverse will primers will hybridize independently to complementary capture oligonucleotides in a feature (i.e., either a forward primer or a reverse primer will hybridize to any one capture oligonucleotide). In other embodiments, the forward and reverse primer pair constructs each comprise a different 5′ terminal linker sequences. In certain implementations of this embodiment, the sequence complementary to the 5′ terminal linker sequence of the forward primer construct and the sequence complementary to the 5′ terminal linker sequence of the reverse primer construct are present but distinct within the same capture oligonucleotide (see FIG. 9 and Example 1). Thus, a single capture oligonucleotide will hybridize to both a forward and reverse primer. In alternative embodiments, the sequence complementary to the 5′ terminal linker sequence of the forward primer construct and the sequence complementary to the 5′ terminal linker sequence of the reverse primer construct are present on different capture oligonucleotides.

In embodiments that employ a hybridization of terminal linker sequence(s) to a capture oligonucleotide on the array, the primer constructs can be released by incubating the hybridized complex under denaturing conditions (e.g., high temperature). In additional embodiments, the primer construct includes a cleavable linker downstream of the terminal linker sequence (see FIG. 1B) that can be cleaved to release the primer constructs from the capture oligonucleotide (described below).

As mentioned above, a domain of a primer construct can have multiple functionalities. Thus, in certain embodiments, the 5′ terminal linker sequence of the forward and reverse primer pair constructs each contain a universal primer binding site, e.g., for next generation sequencing, where in some instances the primer binding site on the forward primer construct is different than the primer binding site on the reverse primer construct.

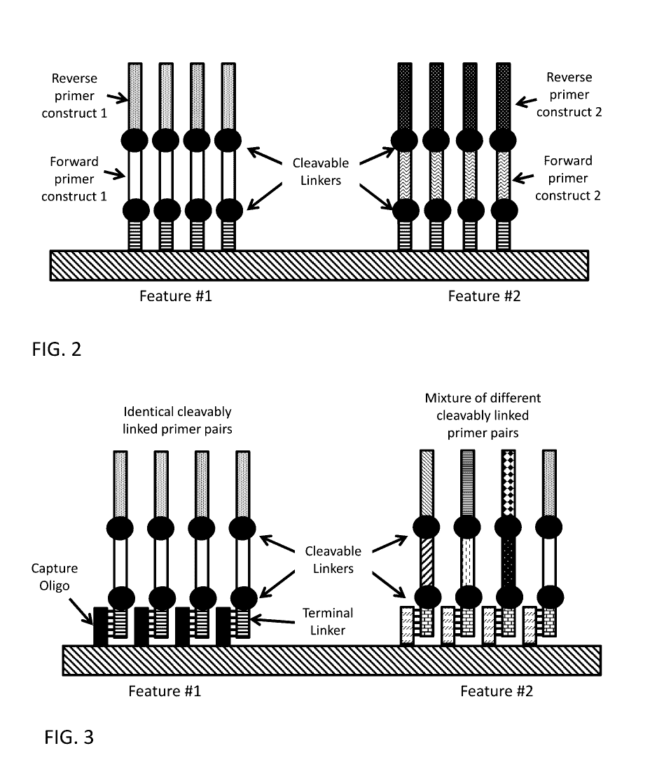

In some embodiments, the forward and reverse primer pair constructs are covalently immobilized to the solid substrate, e.g., via a cleavable linker (see FIG. 2). Release of the primer constructs can be achieved by cleaving the cleavable linker prior to or during the incubating step. Any of a number of cleavable linkers and methods for their cleavage can be employed. One simple cleavable linker, and perhaps the most practical, is a photocleavable linkage between oligonucleotide segments. Chemically cleavable linkers, such as the one used in Agilent's OLS synthesis, may also be employed. Agilent's cleavable linker is cleaved by ammonia, which may negatively affect enzymatic activity and thus should be used with this in mind. A useful cleavable linker is one that leaves a hydroxyl on the 3′-terminal on the oligonucleotide sequence. Some chemistries leave such a 3′-terminal. Another alternative means of cleavage is with a restriction endonuclease, e.g., one with an offset cutting enzyme (e.g., a Type IIS restriction enzyme). This approach is less efficient in terms of the length of the oligonucleotide sequences required, since either an additional set of oligonucleotides will need to be added and hybridized to the oligonucleotides to be cleaved, or the primer precursor oligonucleotides will need to be folded into a hairpin structure with double stranded parts of a stem at the positions to be cut by the enzyme. This approach generates unused fragments as well as the desired primers. This enzymatic cleavage reaction can be delayed until after droplet formation and coverage by use of temperature (discussed below), where the droplets are kept cold (below the activity temperature of the restriction enzyme) until such time at the digest of the long oligonucleotides is needed.

Photo-cleavage is enabled by incorporation of a photo-cleavable linker into each primer (or primer precursor) oligonucleotide sequence during synthesis (primer precursor is an oligonucleotide that includes a primer/primer construct and at least one additional nucleotide sequence that is cleaved from it to produce the primer/primer construct that is used in the PCR reaction). Photo-cleavage is accomplished by illumination of the oligonucleotide with an ultraviolet (UV) source, such as a mercury lamp, LEDs, or UV laser through a glass substrate (or slide) or from above, e.g., through the oil layer that contains the droplets. Photo-cleavable linkers include phosphoramidites that are commercially available from companies such as Glenn Research. These can produce oligonucleotides that have hydroxyls at their 3′-ends making them good substrates for polymerases used in PCR.

Photo-cleavage of linkers enables the spatial isolation of individual features or droplets, allowing the ordering of the release of oligonucleotides to be controlled. This means for example that internal primers can be released before external primers, further controlling nested PCR or staged amplification. Spatial control of the release of photocleavable linkers is accomplished by means of a UV source combined with a microscope objective or other light scanning system, where the source may be a laser, a UV lamp, an individual LED, or an LED or Diode laser array.

In some applications, the forward and/or reverse primer construct will include a barcode sequence (see FIGS. 1A, 1B and 1C), where the barcode sequence is positioned upstream of the target nucleic acid binding site and downstream of a universal primer site, when present. The design and use of barcode sequences is within the skill in the art. In general, nucleic acids in different samples can be tagged with different barcode sequences such that each barcode is indicative of the sample origin of a nucleic acid (the nucleic acid in sample 1 is tagged with barcode 1, the nucleic acid in sample 2 is tagged with barcode 2, etc.). It is thus possible to identify the source of a target nucleic acid by determining its barcode sequence, even when samples are mixed. Bioinformatic methods for determining the source of a nucleic acid based on a barcode sequence are commonly referred to as deconvolution.

For most applications, a barcode sequence need only add from about 4 to about 10 additional bases in a primer construct. For example, only four additional bases are necessary to generate 256 different barcodes to label 256 different samples while a barcode of 10 base pairs can distinguish up to 1 million distinct samples. However, it is often useful to make barcode sequences redundant to avoid low complexity sequences, and to avoid genomic sequences of the organism of interest, which may lead one to use barcodes that are somewhat longer than may minimally be necessary. Longer barcode sequences can provide redundancy that enables error correction methods known to those skilled in the art of information theory. In many applications, only one primer of each of primer pair needs to include a barcode sequence to achieve sample barcoding, although a second barcode, either identical to or different from the first barcode, can be used to provide additional information, such as sample labeling redundancy.

In some embodiments, the barcode sequence is a degenerate barcode sequence. It is noted here that in certain embodiments, the barcode sequence is not completely degenerate, and thus different barcodes can differ from each other by only a subset, but not all, of the positions within the barcode sequence (e.g., at one, two, or three positions in a 10 nucleotide barcode sequence). Moreover, some variable positions in the barcode sequence can be partly degenerate, for example differing across a subset by only 2 or 3 of the 4 canonical nucleotides at each degenerate position.

Degenerate barcode sequence differences can be generated during synthesis using “degenerate base” sites, where each degenerate base is a specific mixture of nucleotides from which different oligonucleotide molecules will have different nucleotides randomly incorporated into the sequence at each degenerate-base position. In some cases, the nucleotide mixtures can be biased, making some nucleotides more abundant than others. Furthermore, degenerate parts can be designed contingent upon how many barcodes are required and can contain substantial constant parts. The fewer barcodes that are required, the fewer degenerate sites needed. As an extreme example, when only 4 barcodes are required, for example a 21-nucleotide degenerate region can contain constant positions 1-10 and 12-21, with only position 11 being degenerate and having random incorporation of G,A,T,C. If 1000 distinct barcodes are required, then as few as 5 bases (4⁵=1024 distinct combinations) can be made polymorphic.

As mentioned above, in certain embodiments, the forward primer construct and/or the reverse primer construct comprises a universal primer binding site, e.g., a sequencing primer binding site for next generation sequencing. In general, universal primer binding sites (sometimes referred to as “sequencing adapters”) are from about 18 to 25, e.g., 20, bases in length.

It is noted here that it is within the skill in the art to design forward and reverse primer pairs that include the desired primer sequence and additional domains that, when employed in downstream processes, will maintain sequences necessary for the desired analysis. For example, positioning the barcode sequence downstream of the universal primer site and upstream of the primer sequence specific for the target nucleic acid to be amplified so that a sequence read will include the barcode sequence.

Given the description herein of the possible different primer construct domains, the size of a single primer construct can be from about 15 nucleotides to about 70 nucleotides, e.g., from about 20 to 50 nucleotides, or more.

In some embodiments, multiple primer constructs are linked together via a cleavable linker (see FIG. 1C and FIGS. 2 to 6). For example, forward and reverse primer pair constructs can be linked to each other via a cleavable linker that is cleaved prior to or during the amplification step (or the “incubating step”) thereby releasing the individual primers. The cleavable linker can be any type of cleavable moiety, e.g., a chemically cleavable linker, a photo-cleavable linker, an enzymatically cleavable linker, and combinations thereof. Any combination of primer constructs can be connected vial cleavable linkers, including one or more sets of forward and reverse primer constructs. Different pairs of forward and reverse primer constructs can be designed to amplify completely distinct target sequences, such as overlapping target sequences, or nested target sequences, as desired. No limitation in this regard is intended.

Thus, certain embodiments include arrays of oligonucleotide features that include first forward and reverse primer pair constructs for amplifying a first target sequence and second forward and reverse primer pair constructs for amplifying a second target polynucleotide sequence. In certain embodiments the first and/or second forward and reverse primer pair constructs are linked by a cleavable linker. In such embodiments, the first and second forward and reverse primer pair constructs can be linked in any desired manner, e.g., with only the first primer constructs linked to each other, with only the second primer constructs liked to each other, with the first primer constructs linked to each other and the second primer constructs liked to each other, or with all of the first and second primer constructs linked to each other (i.e., all four primer constructs are linked into a single oligonucleotide component). In certain embodiments, the amount of the first forward and reverse primer pair constructs in the feature is less than the amount of the second forward and reverse primer pair constructs in the feature. In certain embodiments, the first and second forward and reverse primer constructs form a nested primer set whereas in other embodiments, the first and second forward and reverse primer pair constructs are for amplifying non-overlapping first and second target polynucleotide sequences.

In certain embodiments, the array includes one or more oligonucleotide features (one, a subset, or all of the plurality features on the array) that have from 2 to 100 different forward and reverse primer pair constructs therein. In some embodiments each oligonucleotide feature on the array, or a subset of the plurality of features, has the same set of forward and reverse primer pair constructs. In some instances, one or more of the plurality of oligonucleotide features on the array has a different set of forward and reverse primer constructs as compared to at least one other oligonucleotide feature on the array.

Microarrays containing a plurality of features containing reversibly-immobilized primer pair constructs can be generated in a variety of ways. For example, oligonucleotide features containing the desired primer pair constructs can be synthesized directly on a solid substrate to generate a microarray, e.g., where each feature contains synthesized oligonucleotides containing cleavably-linked primer pair constructs. As another example, an oligonucleotide microarray with features containing a panel of capture oligonucleotides (indexing sequences) can be generated and then contacted under hybridization conditions to a pool of primer construct oligonucleotides with terminal linker sequences that target them to a desired location on the array (via hybridization to the desired capture oligonucleotide), e.g., an Oligo Library Synthesis (OLS) library (Agilent Technologies). Thus, using these or other array fabrication approaches, one can collect a high number of distinct primer pair constructs in each oligonucleotide feature on solid support. It is noted that in some instances, in order to accomplish barcoding geometrically, specific features on an array can be given indexing sequences associated with distinct samples, where each distinct indexing sequence is associated with a distinct barcode.

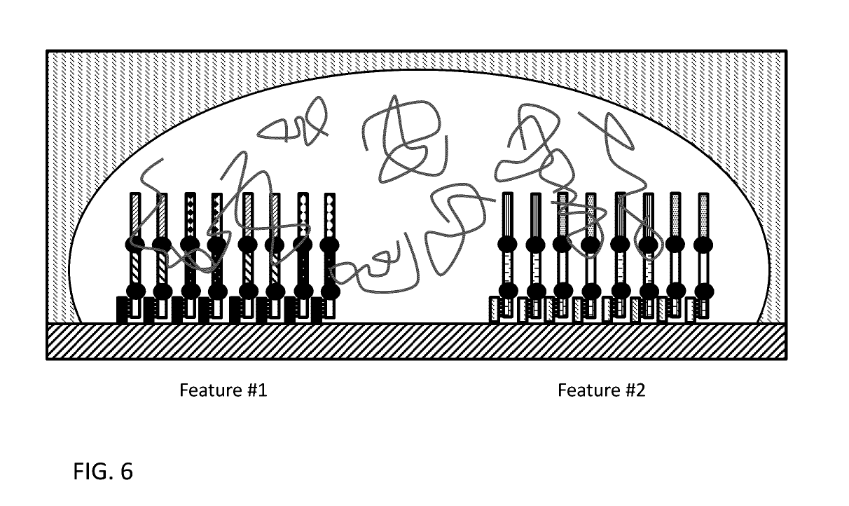

Once a solid substrate containing a plurality of oligonucleotide features (array) is provided, nucleic acid samples can be contacted to each of the features on the array. This contacting can be achieved in any convenient manner. In certain embodiments, a sample can be deposited onto the surface using an ink-jet, or a valve-jet nozzle, or a pipette, such as in a robotic system, or fired by an acoustic wave from a well-plate opposing the array, such as Labcyte's Echo™ liquid handling system. To slow or halt the evaporation of sample droplets during deposition, the time in which to deliver sample may be extended by means of a humidity-controlled environment, which keeps the relative humidity above 90%. Evaporation time can also be increased by means of reagents that include glycerol or DMSO, which have lower evaporation rates than water. In some embodiments, a set of adjacent features on the array (a cluster) is contacted with a single sample. In certain embodiments, e.g., with a deposition approach, sets of adjacent features can be arranged in clusters and a relatively large droplet of a single nucleic acid sample can be deposited to contact a number of the adjacent features in the cluster simultaneously, for example by a robotic pipetting system (e.g., as shown in FIG. 6). Alternatively, the droplets can be fired at one or more central features within a cluster until the cumulative droplet exceeds the boundary of the first feature and expands to include the other adjacent features surrounding the central feature(s). In this case, and where the features are arranged in a hex-pack configuration, sets of features for a sample are arranged in concentric rings of 7, 19, 37, etc., features, and separated from each other by at least one, or more than one, feature spacing. Similarly, other geometries are possible. In certain embodiments, one or more of the plurality of features on the array is contacted with a different nucleic acid sample as compared to at least one other feature.

The nucleic acid samples contacted to the features of the array can differ in any way desired by a user, e.g., from different individual cells, different tissues, different subjects, etc. In some embodiments, the different nucleic acid samples each include a single nucleic acid molecule (e.g., a chromosomal fragment), whereas in other embodiments, each of the different nucleic acid samples includes multiple nucleic acid molecules. Thus, in certain embodiments, the target sample may contain/consist of a pool of target molecules, a single multi-cellular sample, a set of distinct samples from numerous cells. Additionally, each sample can contain the DNA or RNA from a single cell, or nucleus, or a single chromosome. These samples may be isolated from each other in droplets. In applications where whole chromosomes, large sections of chromosomes or long fragments are isolated together in a single droplet, haplotypic phasing can be achieved between amplicons within the same fragment within the same droplet. No limitation in this regard is intended. In this way, structural variations, such as chromosomal rearrangements, translocations, inversions, and gene fusion events can be observed.

Delivery of the nucleic acid samples can be achieved in any convenient manner. For example, microfluidic channels can be used between two planar surfaces (one surface being the array, the other being microstructures) to guide each nucleic acid sample to its corresponding feature, where in some cases the feature is encased in an aqueous droplet. Samples can be moved to the appropriate features by means of pressure differentials, or positive displacement. In another embodiment, the motive force can be applied to droplets by optical tweezers or electrowetting. In yet another embodiment, a pattern of nucleic acid sample droplets with a pattern that is complementary to the pattern of features e.g., in droplets, are brought into physical contact by moving two substrates together from opposite directions. No limitation in this regard is intended.

In some embodiments, the nucleic acid sample introduced to a corresponding feature contains whole live or dead cells. In these embodiments, the cell membranes (outer wall and nuclear membrane) can be lysed by a number of distinct treatments, known to those skilled in the art. These treatments include the application of a lysing solution (usually basic) followed by the application of a neutralization solution. These reagents can be introduced to the sample droplets by the same means of applying the sample to the feature droplet. Another method for cell lysis is the application of high-power laser pulses that deliver heating energy to the cell causing cavitation and localized damage near and within the cell. Once the cell is lysed and the solution neutralized, a master mix of PCR amplification reagents can be deposited to amplify the desire target nuclei acid(s) with the forward and reverse primer constructs in the feature.

In certain embodiments, the array feature and nucleic acid samples are combined as separate aqueous droplets present on independent solid substrates. Once combined, the droplets can be manipulated to amplify the target nuclei acid as desired. In some embodiments, the combine droplets are harvested for further analysis/processing after release of the primer constructs from the array feature. In some embodiments, the droplets can be harvested from the substrate(s) by: a) moving a hydrophobic blade across the surface of the substrate(s), i.e., gently scraping the droplets off the surface of the substrate(s) using a blade; b) displacing the droplets laterally by applying a liquid shear force, e.g., by gently wiggling the array from side to side; c) displacing the droplets by centrifugation; d) causing the droplets to expand, e.g., using heat or negative pressure; e) cleaving the oligonucleotides to which the transposase complexes are hybridized (e.g., via a cleavable linker); f) firing a shaped acoustic or ultrasonic wave or pulse at the droplets; or g) aspirating the droplets from the surface using an aspirator that has a set of channels aligned with the droplets. Depending on the density of the droplets relative to the immiscible liquid, the droplets can rise or fall into the substrate.

The general process of aqueous droplet formation and harvesting (e.g., in emulsions) can be found in co-pending U.S. patent application Ser. No. 14/684,028, filed on Apr. 10, 2015 and entitled “Creating and Harvesting Surface-Bound Emulsion”, hereby incorporated by reference herein in its entirety.

As indicated above, the amplification reaction can be performed by applying a master mix of amplification reagents to each of the plurality of oligonucleotide features. In some embodiments, applying the master mix is performed before contacting with the nucleic acid sample, whereas in other embodiments, the applying the master mix is performed after contacting with the nucleic acid sample. The master mix can include any reagents necessary for amplification, including but not limited to: one or more polymerases (e.g., thermostable polymerases), buffering agents, and deoxyribonucleotide triphosphates (dNTPs) required for polymerization, etc.

Application/deposition of the sample and reagents to each feature leads to the formation of droplets on the array surface in air. In some instances, the droplets can include surfactants or chemical agents to modify the viscosity of the fluid in the droplet. Viscosity modifying agents include, but are not limited to: glycerol, ethylene glycol, polyethylene glycol (PEG), hydrogels, alginates, and the like. Some of these are known to those skilled in the art of inkjet printing, which also puts constraints on the viscosity of solutions that can be fired by ink jets.

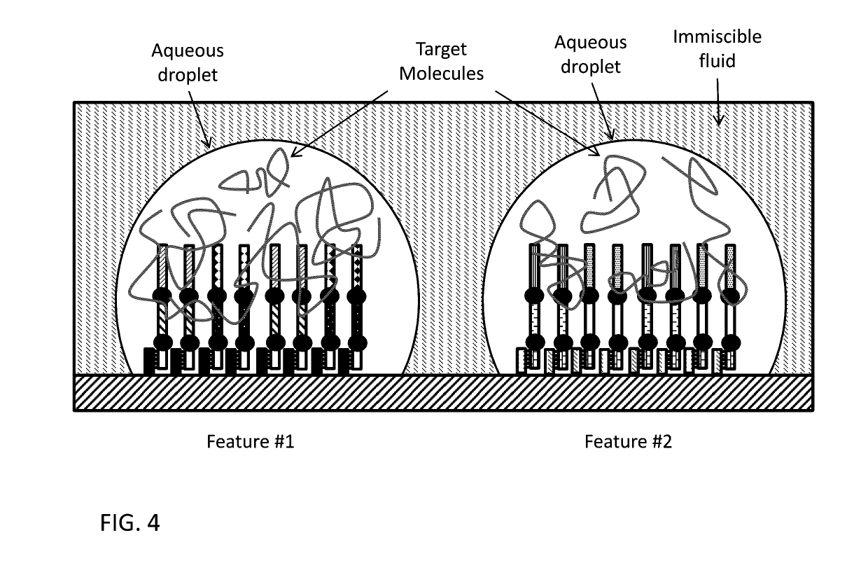

In some embodiments, the droplets on the array are immersed in an immiscible fluid once the sample and reagents have been deposited (contacted) to each of the features (see FIGS. 4, 5, and 6). This immersion step can protect the droplets from evaporation. Once immersed, the array can be incubated under conditions for performing the amplification reaction, e.g., by varying the temperature through a series of thermal cycling steps necessary for performing a desired PCR. The immiscible liquid can comprise a mineral oil such as Petroleum Special, an alkane such as heptadecane, a halogenated alkane such as bromohexadecane, carbonated oils, perfluorocarbon oil, e.g. 3M's Novek™ HFE-7500, an alkylarene, a halogenated alkylarene, an ether, or an ester having a boiling temperature above 100° C. (e.g., above 130° C.), for example. The immiscible liquid should be insoluble or only slightly soluble in water.

Application of the oil to the array can be done in any convenient manner, e.g., by dipping the slide in oil, by filling a chamber containing the slide with oil, or by exposing the slide to oil vapor and condensing the oil on the slide. Fluorinated oils, such as Novek-7500 can be used, as they can have desirable boiling points and have high vapor pressures, making clean up simple. Surfactants can be used to change the stability of the droplets and control the droplet formation. Useful surfactants and detergents include but are not limited to: fluoro-surfactants, such as Picosurf, PEG, Perfluorooctanesulfonic acid (PFOS), siloxane surfactants, ionic surfactants, Tego, and Triton X, NP-40. Certain surfactants include hydrocarbon chains, fluorocarbon chains, and/or a hydrophobic head and a hydrophilic tail.

An example of the process for performing on-array multiplex PCR is depicted in FIGS. 4, 5 and 6, and described below.

A first array is fabricated with oligonucleotides in such a way that each contains capture oligonucleotide (or indexing) sequence and one or more primer constructs hybridized thereto via their terminal linkers. As shown in FIG. 3, the oligonucleotides hybridized to the indexing array can be long oligonucleotides with photocleavable linkers that separate primer constructs (oligonucleotides without cleavable linkers may be used; see Example 1). In some embodiments, a feature can have identical cleavably linked primer constructs (e.g., primer pairs; Feature #1 in FIG. 3) or can have a mixture of different cleavably linked primer constructs (e.g., multiple different primer pairs; Feature #2 in FIG. 3). It is noted here that a single oligonucleotide can have more than two primer constructs linked by cleavable linkers, including 3, 4, 5, 6, 7, 8, 9 or 10 primer constructs having any combination of specific sequences (e.g., identical copies of a single primer construct, multiple different primer constructs, or a combination thereof). No limitation in this regard is intended.

The arrays are exposed to a master mix solution containing: polymerase, buffer, dNTPs, fluorescent markers, such as molecular beacons, and salts, or cofactors that ensure proper function of the enzyme. This step can be performed in a flow cell by filling, and draining the cell with the master mix and leaving behind droplets on the hydrophilic oligonucleotide features (as described in a previous patent application). For a single (non-multiplexed) sample, the DNA can be included in the master mix before flooding.

For the case of individualized samples, such as contents of single cells, the arrays droplets on the array are exposed to additional droplets with sample, such as a DNA sample, a sample of RNA, whole chromosomes, or plasmids. These droplets can be deposited directly on top of the surface, or deposited with other fluid mechanical means.

The droplets can be immersed in an encapsulating fluid, or oil, to encapsulate each droplet on the surface of the slide, thus preventing drying or mixing of samples, as shown in FIG. 4.

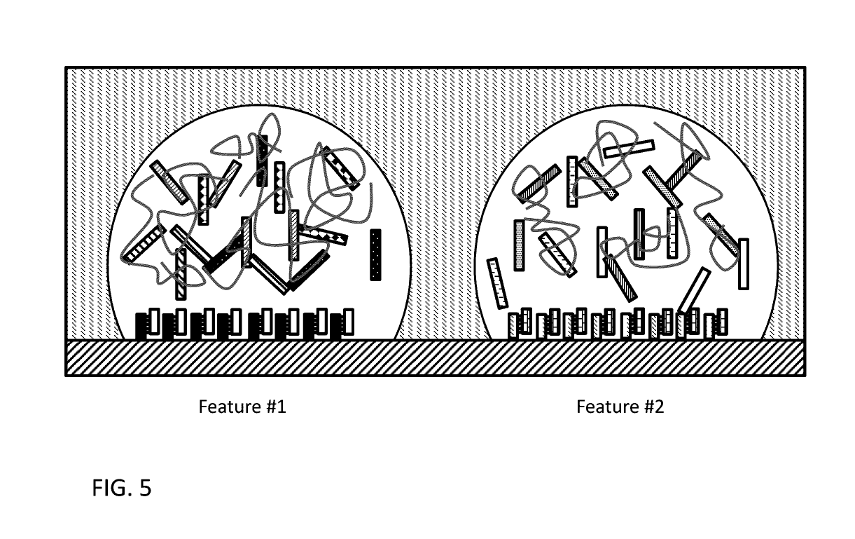

The slide is exposed to light, usually ultraviolet (UV) light, that cleaves oligonucleotides at the cleavable linker sites (FIG. 5). In some embodiments, a chemically cleavable linker is used, where the chemical that cleaves the linker is introduced by diffusion, or added with the sample. In these embodiments, the chemical must not alter the activity of the enzyme.

Once the flow cell is sealed, the temperature of the cell is cycled through multiple temperature cycles of PCR, e.g. 10 to 40 cycles over a temperature range typically from about 65° to about 95° C. This process may be monitored in real time by an optical fluorescent microscope or imaging system. If so, quantitative PCR can be achieved.

Once amplified, the products can be harvested by aspirating the carrier oil from the flow cell, and eluting the product by refilling the flow cell with an aqueous reagent, e.g. a buffered aqueous reagent with a surfactant, and then aspirating aqueous fluid from the cell.

In another embodiment, the incubating step includes performing a real time PCR. In certain of these embodiments, a labeled reporter molecule, such as a molecular beacon or an intercalating dye, can be included into the master mix or target solutions. This reporter molecule provides a signal within each droplet when the appropriate target is amplified. In this embodiment, an excitation from a light source can be applied to the slide with droplets in oil during amplification. A fluorescent signal would thus be produced in real-time, during amplification that would interrogate the droplet for the quantity of specific DNA product produced in each droplet. Using molecular beacons specific to targets between primers, different targets can have differently labeled beacons, making multiple distinct products discernable within a single droplet. This embodiment utilizes a light source, excitation filters, emission filters, an objective lens, and an optical detector in configuration of a fluorescent microscope or microwell-plate reader.

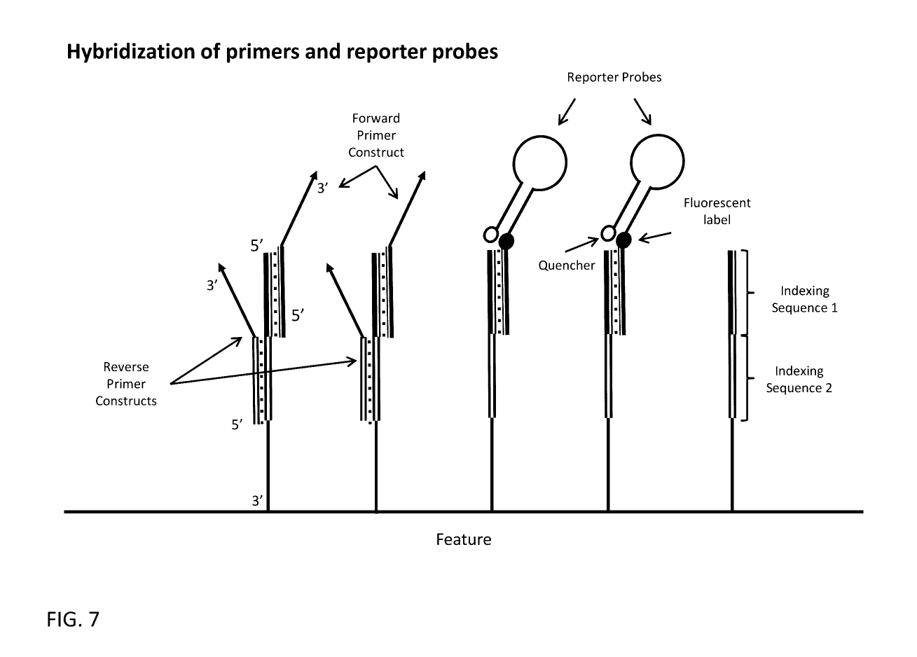

In another embodiment, a reporter probe or molecular beacon, which includes an indexing sequence, is used to quantitatively report on a target of interest. This reporter probe consists of an indexing sequence and a labeled oligonucleotide where the fluorescence is quenched in the absence of target and unquenched when hybridized specifically to the PCR amplicon (such as a molecular beacon), or when the exonuclease activity cleaves and releases either the fluorescent label or its quencher from the probe (such as with a TaqMan probe). In this embodiment, as depicted in FIG. 7, reporter probes having a fluorescent label and quencher moieties (filled and open circles in FIG. 7) are hybridized to the features along with the primer constructs prior to contacting the sample to the array. The sequence within the reporter probe targets the amplicon sequence. Then, during the annealing or extension cycles of PCR they can be used to report on the quantities of the amplified targets of interest in each droplet. In this way, multiplex quantitative PCR can be performed in a on a feature-by-feature basis. Multiple distinct reporters with distinct fluorescent labels can be used to address different targets within each droplet.

In an approach utilizing only a single microarray, we can produce between one and ten distinct oligonucleotide sequences per oligonucleotide feature by means of DNA synthesis followed by cleavage of each oligonucleotide using a cleavable linkers, positioned at numerous sites within each synthesized long oligonucleotide. Agilent's current long-oligonucleotide chemistry can efficiently produce 200 bp oligonucleotides with a yield of approximately 8,000 full-length oligonucleotides per square micron within a feature, and even longer oligonucleotides have been achieved, with somewhat reduced yield. For features with a diameter of 50-microns, this density yields roughly 16 million long oligonucleotide molecules per feature. If each OLS microarray contains 250 k features, then from 1 million to 2.5 million distinct primer constructs can be produced from a single microarray, where each construct is represented by approximately 16,000,000 molecules. By combining the oligo complexities of OLS libraries with the feature indexing of a microarray, there is quite a bit of flexibility as to the numbers of distinct primer populations that can be hybridized to each feature. From as few as a single primer pair per feature to as many as hundreds of thousands of primer pairs per feature can be accommodated, and where no two features have the same mixtures of primer pairs. The primer construct hybridization method allows for great assay flexibility. The readouts can be either quantitative in real-time, or discrete end-point measurements, like droplet digital PCR.

By way of example, two different embodiments of the present disclosure are provided below. The first involves the use of multiple features per target sample, and the second involves the use of an indexing array hybridized with an oligonucleotide library having terminal likers.

In the first approach, (a “single-array multiple-feature approach”) an aqueous droplet containing sample or a mixture of target molecules encompasses one feature or multiple features. The pooling of oligonucleotides can be done in several ways: in one method a fluid containing master-mix is flooded onto a microarray slide, and then eluted to produce one droplet per oligonucleotide feature. These droplets can be allowed to dry on the array features or remain hydrated as small droplets on its surface. Subsequently, a sample droplet (or set of droplets) including target molecules is (are) contacted or deposited on top of a feature or set of features on the surface, described further below. Once the sample is applied to one or more features, the oligonucleotides on the surface of the feature can be cleaved (either chemically or by photocleavage of a cleavable linker) releasing multiple primers into the droplet or compartment on the surface of the substrate. Alternatively, the oligonucleotides can be pre-cleaved, for example by exposure to ammonia, before the application of the sample.

Each oligonucleotide feature is comprised of an oligonucleotide sequence with at least one primer sequence and a cleavable linker. Each oligonucleotide may comprise a single primer or multiple primers (see FIGS. 1A, 1B and 1C). An array with two distinct features, where each feature consists of two primer constructs separated from the adjacent constructs and from a linker sequence by cleavable linkers, is depicted in FIG. 2.

In the second approach, (an “indexing array+OLS approach”) an indexing array is used to pull oligonucleotides from a solution containing an oligonucleotide library (OLS) down to a DNA-array feature by means on an indexing sequence at the end of each oligonucleotide in the library that is complementary to the feature sequence (see FIG. 3). In this way, each feature can pull down a distinct predefined set of oligonucleotides and hold them on the surface by hybridization. Different features can have different oligonucleotide sets or they can share some or all oligonucleotide sets. In principle, each feature can pull down thousands of distinct oligonucleotide primer pairs (see Feature #2 in FIG. 3). In this way, a whole array can have a great number of target sets in distinct features. In practice, the number of distinct pairs is limited by the practical considerations of oligonucleotide density, feature size, target number, and amplification gain needed per feature. The higher the number of distinct primer pairs needed, the smaller the number of each pair that can be pulled down by hybridization to each feature. Thus, a 50-micron feature hybridized to 1000 primer pairs would have about 10,000 of each primer pair, thus limiting amplification accordingly to approximately 11 or 12 cycles of PCR for a single-single molecule target. This limitation may be a good match for single-cell sequencing applications, where each cell has two genomic targets and limited, uniform, amplification gain from cell to cell is beneficial for efficiently sequencing a number of cells.

Each oligonucleotide of the OLS library may comprise multiple distinct or identical primer constructs, as shown in FIG. 3, where each oligonucleotide starts with an indexing sequence, and contains more than one primer construct, with a primer sequence and possibly a sequencing adapter and/or a barcode sequence. Each of these primer constructs (adapter+barcode+primer) is separated from the next, and from the indexing sequence, by a cleavable linker. This embodiment has the virtue that all primers that are in the same original oligonucleotide sequence will be present on the feature and in the droplet with the same copy number. This is ensured by the 3′ to 5′ synthesis of the oligonucleotides with the indexing sequence at the 5′ end of the oligonucleotide. So if the sequence terminates early during synthesis, the remaining sections should be present in the absence of post-synthesis oligonucleotide cleavage.

All the primer constructs within an oligonucleotide or a feature are not necessarily identical, although in some embodiments that may be advantageous (as shown in Feature #1 in FIG. 3). To balance the forward and reverse primer concentrations in the each droplet, it may be useful to design oligonucleotides in such a way that that the forward and reverse primers for each target are synthesized within the same oligonucleotide construct.

Another benefit of this approach is that since all oligonucleotides for a given target mix are hybridized with the same indexing sequence, all primer constructs should hybridize to each feature with approximately the same efficiency, thereby equalizing the number-density on the surface according to the concentration of each in the hybridization mix (assuming a similar number of capture oligonucleotides comprising each feature). This means that the OLS library should produce a uniform concentration for all primer oligonucleotides. As such, it may be beneficial to either (1) produce libraries with internally redundant array designs, where each oligonucleotide is produced at numerous positions (or swaths) within the same array, or (2) to do the same variation across different OLS array designs, where each design has the same oligonucleotide content but different array layouts. The most robust assay would likely come from doing both.

In this general indexed hybridization-based approach, if capping is used in the oligonucleotide synthesis process, then only those sequences without coupling errors will achieve full length, and only those full-length oligonucleotides will have 5′ ends that can hybridize to the indexing array. This also means that each oligonucleotide, once cleaved will generate exactly the same number of primer constructs.

It is noted that in certain embodiments, the single array and indexing array embodiments above can be combined.

In another application, each feature can contain primer pairs for distinct exons of a distinct gene or genomic locus. In this way, amplicons across a genome or exome can be mapped out geometrically on an array. Or, allele-specific primers can be introduced making possible multiplex genotype calling, without the need for sequencing the product.

For a pool of 1,000 or more cells, it may be necessary to expose the sample to multiple, perhaps hundreds, or even thousands of array features, to provide a sufficient number of primers for significant amplification. If 1000 samples are amplified on a single slide, then approximately 10-1000 features are available to achieve sufficient amplification, depending on how many targets are to be amplified within the panel and the plexity of labeling, or whether the readout is sequencing. The number of samples and the number of targets will determine which of the two embodiments, described above, is more practical.

One benefit of the array-indexing approach is that the concentrations of the starting oligonucleotides can be carefully balanced, and the numbers of primers in each droplet can be made constant. This means that less biased amplification can be achieved in droplets than in bulk, as long as the droplet volumes, surface areas, and oligonucleotide surface densities can be kept relatively constant.

Since each droplet provides a separate reaction vessel there is only competition for resources (such as enzyme molecules and nucleotides) within a droplet, making amplification more uniform than it would be in bulk.

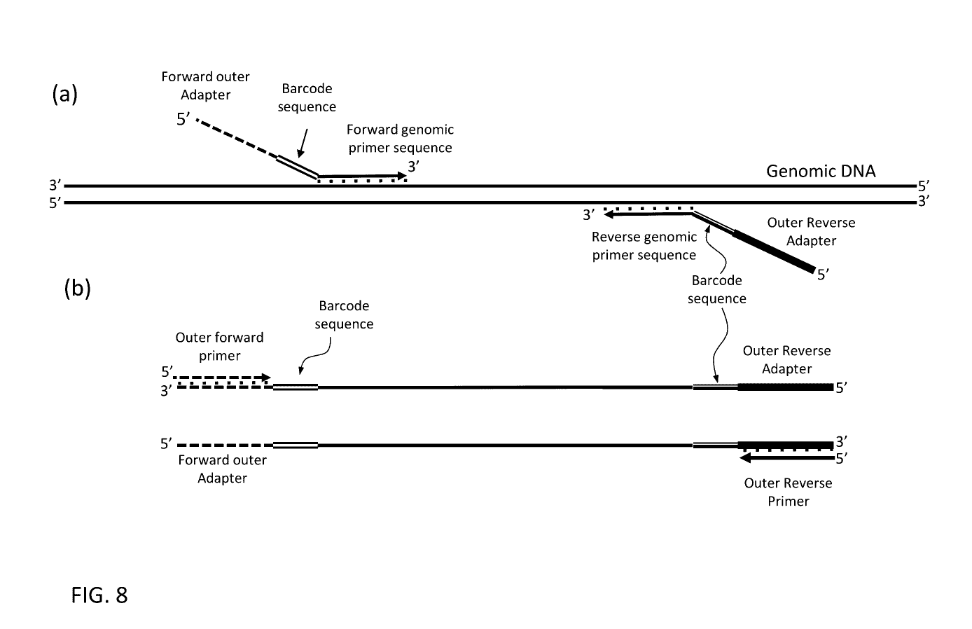

In some sequencing applications it is useful to measure the amplification bias of each product or allele by differentiating between the molecules copied or amplified early in the amplification process and those subsequently amplified. By doing this, amplification biases can be measured and corrected for computationally (termed “amplification bias suppression”). This can be accomplished by using nested primers, where an inner set of primers is used to copy the original genomic material once, or perhaps a small number of times. This early stage amplification is followed by adding more cycles of PCR using an outer set of primers that target adapter sequences included in the first stage primer constructs. In these applications, in addition to the barcodes described previously, an additional random molecular barcodes can be incorporated by means of random bases written into the barcoded oligonucleotide primer construct in the first or earliest PCR cycles. This is accomplished by designing oligonucleotides with two pairs of nested primers, where between the inner primer pair and the outer primer pair is a random-nucleotide barcode, which can be including in either the forward or the reverse genomic direction. The inner pair of primer sequences targets a genomic target sequence and the outer primer pair amplifies the product of the first amplification step using exogenous primer sequences that are complementary to the outer portions of the internal primer constructs, as depicted in FIG. 8. These sequences are usually common across multiple targets, and often used as sequencing adapters. The inner set of primers is thermally cycled a relatively low number of times, typically from 1 to 10 thermal cycles. Additional amplification can be had, as necessary, from the set of outer common primers.

In one primer construct, either at the 3′-end or 5′-end of the genomic target, or both ends, barcodes can be incorporated between the inner and outer primer sequences. It may be useful to use two distinct barcodes, or random-base barcodes. Random-base barcodes (also known as “degenerate-base” barcodes) are random on the single molecule scale, and can be generated using a mixture of nucleotides incorporated into the construct during DNA oligonucleotide synthesis on a microarray, or on solid support. Non-random barcodes are common to many (if not all) oligonucleotides within a feature or set of features.

In some applications, it may be useful to suppress further amplification with the inner primers, while allowing amplification with the outer. Two approach can be used for this control: the first is to limit the number of internal primer sequences in the pool so that they will simply run out after the first stage of amplification, and the second is to use a higher annealing temperature for the second stage amplification that the first, so that the inner primers will simply not anneal at the higher annealing temperatures used in a second stage. To accomplish the latter, the outer primer set must have significantly higher melting temperatures than the internal primer sets. This means that the primer lengths for the second-stage primer pairs are designed to be longer than the inner genome targeting pair. So, for example, if the genome targeting sets are 16-20 bases long, the secondary set will likely be 25 bases and longer.

For the method to be applied to single-molecule sequencing, the number of steps in the first round of amplification must be very low (1-4), and the number of random bases should provide sufficient complexity to cover the number of copies of each original genomic target molecules. For genomic sequences, this depends on how many cell equivalent molecules are in each pool. For transcript sequences (transcriptome), this depends on the expression level of the transcript. Single-molecule sequencing makes possible the effective reduction of amplification noise, as has been demonstrated in several papers: Islam et al. “Quantitative single-cell RNA-Seq with unique molecular identifiers”, Nature Methods 11 No 2, p163-166, 2014, and Schmitt et al. “Detection of ultra-rare mutations by next-generation sequencing”, Proc. Natl Acad. Sci. USA 109, 14508-14513 (2012).

Another aspect of the present disclosure includes methods of sequencing target polyncleotides, the method including: performing a multiplex amplification of target polynucleotides according to method disclose herein (e.g., as described above) to produce a pool of amplified target polynucleotides; and sequencing the pool of amplified target polynucleotides. The sequencing can be performed in any convenient manner and may be done using sequencing primers specific for nucleic acid sequences in the forward and reverse primer constructs used to amplify the target polynucleotides. In certain embodiments, the sequencing primers are universal primes, e.g., sequencing primers for next generation sequencing.

Another aspect of the invention relates to oligonucleotide arrays as detailed herein and kits containing them. Thus, aspects of the present disclosure provide an oligonucleotide array for multiplex amplification of target polynucleotides, the array containing: a plurality of oligonucleotide features, wherein each of the plurality of features comprises substrate-immobilized forward and reverse primer pair constructs comprising target nucleic acid binding sites for amplifying a target polynucleotide sequence.

In certain embodiments, the forward and reverse primer pair constructs are immobilized via hybridization of a terminal linker sequence (e.g., 5′ or 3′ terminal linker sequence) to a complementary sequence on a capture oligonucleotide covalently attached to the substrate. In certain embodiments, the forward and reverse primer pair constructs each comprise different terminal linker sequences. In certain embodiments, the sequence complementary to the terminal linker sequence of the forward primer construct and the sequence complementary to the terminal linker sequence of the reverse primer construct are present on the same capture oligonucleotide. In certain embodiments, the sequence complementary to the terminal linker sequence of the forward primer construct and the sequence complementary to the terminal linker sequence of the reverse primer construct are present on different capture oligonucleotides. In certain embodiments, the terminal linker sequence of the forward and reverse primer pair constructs are different universal primer sites. In certain embodiments, the forward and reverse primer pair constructs are covalently immobilized to the substrate. In certain embodiments, the forward and reverse primer pair constructs are linked by a cleavable linker to the substrate, wherein the cleavable linker is cleaved prior to or during the incubating step. In certain embodiments, the forward and reverse primer pair constructs are linked to each other via a cleavable linker, wherein the cleavable linker is cleaved prior to or during the incubating step. In certain embodiments, the cleavable linker is selected from the group consisting of: a chemically cleavable linker, a photo-cleavable linker, an enzymatically cleavable linker, and combinations thereof. In certain embodiments, the forward primer construct and/or the reverse primer construct comprises a universal primer site. In certain embodiments, the forward primer construct and/or the reverse primer construct comprises a barcode sequence upstream of the target nucleic acid binding site and downstream of the universal primer site, when present. In certain embodiments, the barcode sequence is a degenerate barcode sequence. In certain embodiments, the oligonucleotide feature further comprises second forward and reverse primer pair constructs for amplifying a second target polynucleotide sequence. In certain embodiments, the second forward and reverse primer pair constructs are linked by a cleavable linker. In certain embodiments, the first and second forward and reverse primer pair constructs are linked by cleavable linkers. In certain embodiments, the amount of the first forward and reverse primer pair constructs in the feature is less than the amount of the second forward and reverse primer pair constructs in the feature. In certain embodiments, the first and second forward and reverse primer constructs form a nested primer set. In certain embodiments, the first and second forward and reverse primer pair constructs are for amplifying non-overlapping first and second target polynucleotide sequences. In certain embodiments, each of the plurality of oligonucleotide features comprises from 2 to 100 different forward and reverse primer pair constructs. In certain embodiments, each of the plurality of oligonucleotide features comprises the same set of forward and reverse primer pair constructs. In certain embodiments, one or more of the plurality of oligonucleotide features comprises a different set of forward and reverse primer constructs as compared to at least one other oligonucleotide feature.

Aspects of the present disclosure provide kits comprising an oligonucleotide array described herein. In general, kits according to the present invention comprise one or more components of at least one of the aspects of the invention described above that is useful for multiplex amplification of nucleic acid samples. The components of the kits can be provided in, or bound to, one or more solid materials. For example, one or more components can be provided in a container, which can be fabricated from plastic materials and formed in the shape of microfuge tubes or sequencing plates (e.g., 84- or 96-wells per plate). Alternatively, one or more components can be provided as a substance bound to a solid support (as described herein).

As mentioned elsewhere herein, the kits of the invention can comprise any number of substances that are useful for practicing a method of the invention. In certain embodiments, the kit includes amplification reagents or a master mix of amplification reagents. Such reagents include, but are not limited to: reagents (including buffers) for lysis of cells, DNA fragmenting reagents (including buffers), PCR reaction reagents (including buffers), immersion fluids, one or more sequencing primers (e.g., for next generation sequencing reactions), sequencing regents, etc. In certain embodiments, the kit includes instructions for performing a multiplex amplification reaction as described herein.

The kits of the invention can be provided at any temperature. For example, for storage of kits containing transposases, adapters, or complexes in a liquid, it is preferred that they are provided and maintained below 0° C., preferably at or below −20° C., or otherwise in a frozen state.

As noted elsewhere herein, components of the kits are provided in containers or on solid substrates. The containers and solid substrates are provided in packaged combination in a suitable package, such as a box made of cardboard, plastic, metal, or a combination thereof. Suitable packaging materials for biotechnology reagents are known and widely used in the art, and thus need not be specified herein.

### EXAMPLES

The following example is put forth so as to provide those of ordinary skill in the art with a complete disclosure and description of how to make and use the present invention, and is not intended to limit the scope of what the inventors regard as their invention nor is it intended to represent that the experiment below is all or the only experiment performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Primer constructs were hybridized to an Agilent 4-pack array, each with approximately 62,000 features with 50-micron diameters using a modified Agilent SureHyb™ Protocol. The concentration of primers was 0.125 micromolar in a volume of about 0.5 mL hybridization buffer. The primer constructs were hybridized to 4 arrays simultaneously for 16 hours at 65° C. It is estimated that this process produced approximately 10 million primer constructs per feature.

Three primer pairs were used to target two distinct plasmid target sequences: one with a single targeted locus and the other with two targeted loci in the plasmid. One plasmid is a green fluorescent protein (maxGFP™ from Lonza) and the other is EK499 (which encodes a donor sequence for IL2RG). (The biology of the targets is not relevant to the demonstration.)

The capture array used largely contained oligonucleotides with a single indexing sequence, where a first portion of the sequence is complimentary an index sequence attached to the forward primers and a second portion of the sequence is complimentary an index sequence attached to the reverse primers, as depicted in FIG. 9. In principle, each feature or set of features could be addressed independently by use of a distinct pair of indexing sequences to address feature or set of features.

The Hybridized oligonucleotide primer sequences are given here (in 5′ to 3′ orientation):

EK499_S0618_FP1_Alt-Index (SEQ ID NO: 1):
CTATACGACTCTAGGCGTGTACCATGTGGT|TGTAAAACGACGGCCAGT
EK499_S0723_RP1_L125 (SEQ ID NO: 2):
CATGCTACGTATCGCAGTGATTGCACAACG|AACCCACTCTGCTTCTGT
GG
EK499_S4453_FP2_L162 Alt-Index (SEQ ID NO: 3):
CTATACGACTCTAGGCGTGTACCATGTGGT|AGTCTCCCAGGTACCCCA
CT
EK499_S4636_RP1 (SEQ ID NO: 4):
CATGCTACGTATCGCAGTGATTGCACAACG|CAGGAAACAGCTATGACC
ATG
pmaxFP_S0539_FP1_Alt-Index (SEQ ID NO: 5):
CATGCTACGTATCGCAGTGATTGCACAACG|CGCAAATGGGCGGTAGGC
GTG
pmaxFP_S0845_RP2_L211 (SEQ ID NO: 6):
CTATACGACTCTAGGCGTGTACCATGTGGT|GATGCGGCACTCGATCTC

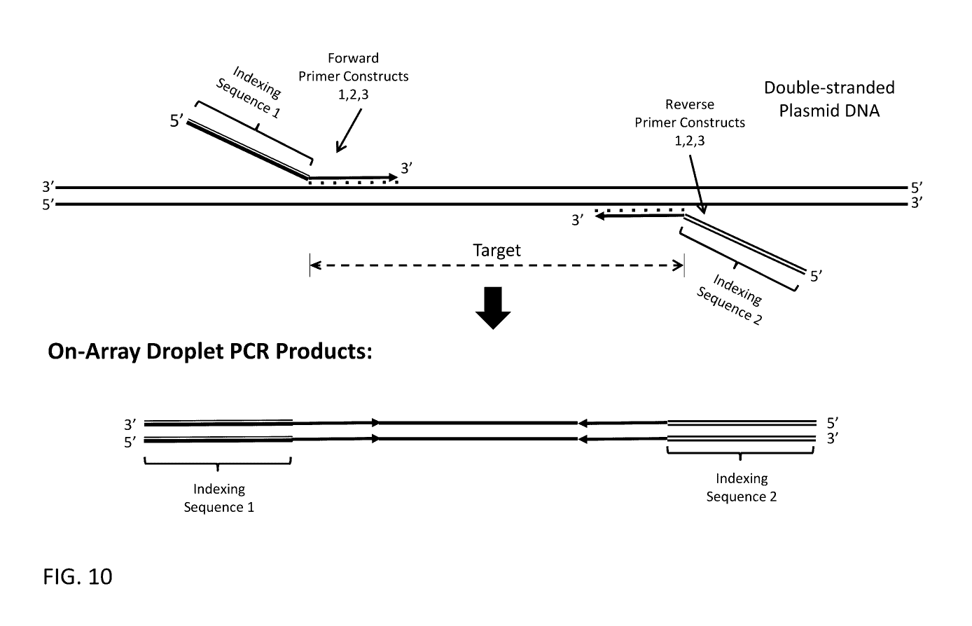

The “I” in the sequences above denotes the position between the hybridized portion of the sequence (underlined) and the primer portion. The geometry of the targeted region is depicted in FIG. 10.

The reaction chamber comprised of two slides was mounted vertically with needles inserted through a gasket between the slides, one at the bottom to fill the chamber and the other at the top to provide a breathing hole to allow air in and out as fluids were added and removed.

The array was exposed to target by filling a well with a reaction mix containing:

| | |
|---|---|
| Template (1 ng/ul) | 24 uL |
| Template (1 ng/ul) | 24 uL |
| 5x Herculase Buffer 0.15% Triton-X-100 | 48 uL |
| 100 mM dNTP | 7.2 uL |
| H2O | 129.6 uL |
| Herculase Enzyme | 7.2 uL |
| Total Volume: | 240 uL |

A volume of approximately 200 uL of this mixture was briefly exposed to two arrays in a single well, by filling the volume made by a 400 micron gap between the two slides (an array and a second blank glass slide). This volume was drained leaving small (50-micron diameter) droplets on the surface of the features of the array. These droplets were imaged using a microscope. The volume was subsequently filled with a fluorinated oil (Fluorinert™ FC-40 by 3M). Filling the chamber with the fluorinated oil serves to stabilize the droplets on the surface by eliminating evaporation.

At this point, PCR was performed by thermally cycling the chamber through 30 rounds of the following thermal cycle:

| | Temp (deg. C.) | Time (s) |
|---|---|---|
| 1. denaturation | 98 | 100 |
| 2. anneal | 57 | 90 |
| 3. extension | 72 | 110 |

These time durations included the temperature transient times, which were from 20-60 s depending on the transition, for a total cycle time of about 5 minutes per cycle.

Because the estimated volumes of the droplets is small, it is estimated that there were approximately 10's of thousands of Herculase™ enzyme molecules in each droplet, and this substantially limited the amount of amplicon product produced in any given droplet to an amount equivalent of between 10 and 15 cycles of PCR. The product DNA was recovered from the reaction reservoir by draining the oil, then refilling the reservoir with about 200 microliters of water. This liquid was concentrated by use of a SpeedVac™ to a volume about roughly 20 microliters. The amplicons were subsequently amplified by a second round of PCR amplification. The second stage amplification used regions within the indexing sequences as primer sites. The indexing sequences were 30 bp long. Two 20-nucleotide primers were made for each indexing sequence: a first was constructed from bases 1 through 20 of each indexing sequences and a second was for bases 11 through 30. The following primers were used for secondary amplification (listed in 5′ to 3′ orientation):

Index 1 primer: index bases 11-30 (SEQ ID NO: 7):
ATCGCAGTGATTGCACAACG
Index 2 primer: index bases 1-20 (SEQ ID NO: 8):
CTATACGACTCTAGGCGTGT
Index 2 primer: index bases 11-30 (SEQ ID NO: 9):
CTAGGCGTGTACCATGTGGT

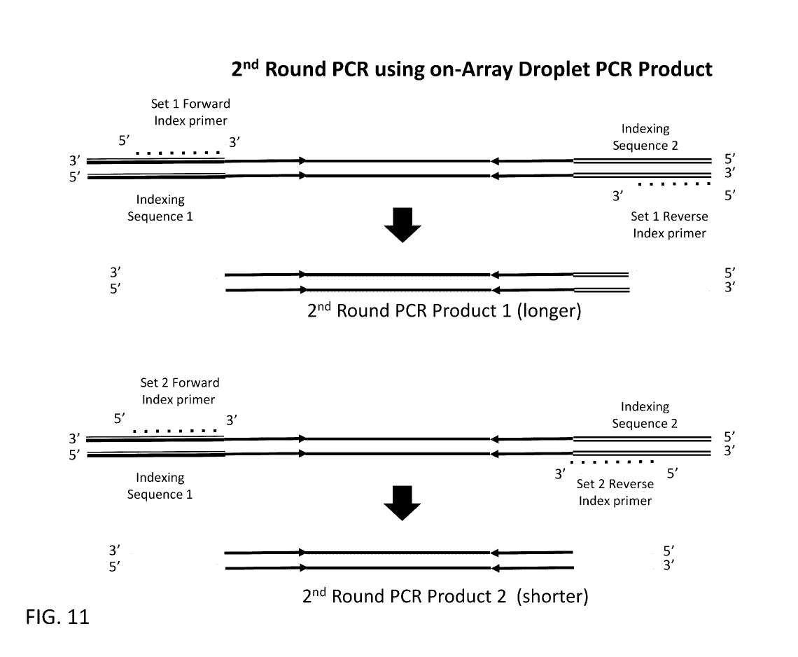

The geometry of the primers within the targeted amplicons is depicted in FIG. 11.

A positive control for all three amplicons was made by amplifying the target vectors in droplets where all reagents, including primers, were included in the mix. These droplets were made by mixing all reagents and running them through a Micronit droplet generator chip that makes mono-disperse droplets approximately 100 microns in diameter. All three targeted amplicons were detected by means of the BioAnalyzer without the use of a second round of amplification. These products were diluted and used as positive controls for second round amplification of the on-array droplet products.

The second round amplification of both the material eluted from the on-array droplet amplification reaction and the positive controls (described above) was performed using a conventional PCR-tube-based thermal cycler for 14 rounds of the following thermal cycle:

| | Temp (deg. C.) | Time (s) |
|---|---|---|
| 1. denaturation | 94 | 30 |
| 2. anneal | 57 | 30 |
| 3. extension | 72 | 90 |

The original vectors were included in the negative control reaction along with the second stage (index-sequence) primers and the master mix, but without the first-stage vector primers. The primers were loaded at a volume of 1.6 ul 100 microM into a total volume of 200 microliters with 2 microliters of 100 mM dNTPs. The Herculase enzyme volume was added at a volume of 2 microliters and 40 microliters of the 5× buffer for that enzyme was used. The sample (after concentration) and positive control volumes were 2 microliters into each reaction.

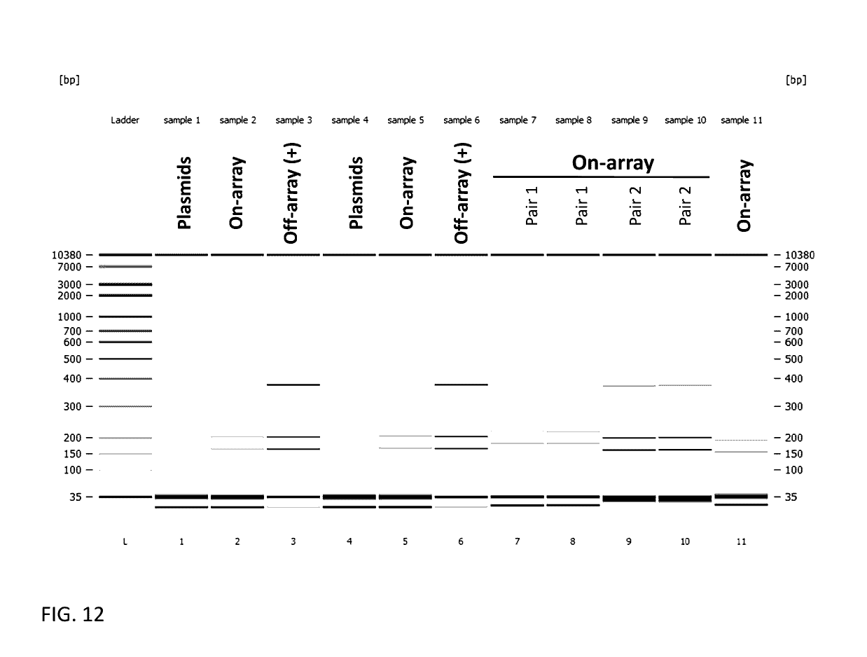

The products of these amplification reactions were run on a BioAnalyzer chip, and the results are shown in the FIG. 12. Different lanes utilized different combinations of primers. Lanes 1-6 and 11 used all three primer pairs, lanes 7 and 8 used primer pair 1, and lanes 9 and 10 used primer pair 2. All lanes showed all three amplicons for both the on-array droplet material and the positive control input material. Differences in product lengths between the use of primer pairs 1 and 2 can be seen in lanes 7-10.

### EMBODIMENTS

A list of non-limiting embodiments of the present disclosure is set forth below.

1. A method for multiplex amplification of target polynucleotides, the method comprising: providing a solid substrate comprising a plurality of oligonucleotide features, wherein each of the plurality of oligonucleotide features comprises a substrate-immobilized forward primer construct comprising a first target nucleic acid binding site and a substrate-immobilized reverse primer construct comprising a second target nucleic acid binding site, wherein the first and second target nucleic acid binding sites are for amplifying a target polynucleotide sequence; contacting each of the plurality of features with a corresponding nucleic acid sample; incubating the contacted plurality of features under conditions to allow amplification of the target polynucleotide sequence by the forward and reverse primer pair constructs, wherein the forward and reverse primer pair constructs are released from the substrate prior to or during the incubating step.

2. The method of embodiment 1, wherein the forward and reverse primer pair constructs are immobilized via hybridization of a terminal linker sequence to a complementary sequence on a capture oligonucleotide covalently attached to the substrate.

3. The method of embodiment 2, wherein the forward and reverse primer pair constructs each comprise different terminal linker sequences.

4. The method of embodiment 3, wherein the sequence complementary to the terminal linker sequence of the forward primer construct and the sequence complementary to the terminal linker sequence of the reverse primer construct are present on the same capture oligonucleotide.

5. The method of embodiment 3, wherein the sequence complementary to the terminal linker sequence of the forward primer construct and the sequence complementary to the terminal linker sequence of the reverse primer construct are present on different capture oligonucleotides.

6. The method of any one of embodiments 2 to 5, wherein the terminal linker sequences of the forward and reverse primer pair constructs are universal primer sites.

7. The method of embodiment 1, wherein the forward and reverse primer pair constructs are covalently immobilized to the substrate.

8. The method of embodiment 7, wherein the forward and/or reverse primer pair constructs are linked by a cleavable linker to the substrate, wherein the cleavable linker is cleaved prior to or during the incubating step.

9. The method of any one of embodiments 1, 2, 7 and 8, wherein the forward and reverse primer pair constructs are linked to each other via a cleavable linker, wherein the cleavable linker is cleaved prior to or during the incubating step.

10. The method of embodiment 8 or 9, wherein the cleavable linker is selected from the group consisting of: a chemically cleavable linker, a photo-cleavable linker, an enzymatically cleavable linker, and combinations thereof.

11. The method of any one of embodiments 1, 2, and 7 to 10, wherein the forward primer construct and/or the reverse primer construct comprises a universal primer site.

12. The method of any one of embodiments 1 to 11, wherein the forward primer construct and/or the reverse primer construct comprises a barcode sequence upstream of the target nucleic acid binding site and downstream of the universal primer site, when present.

13. The method of embodiment 12, wherein the barcode sequence is a degenerate barcode sequence.

14. The method of any one of embodiments 1 to 13, wherein the oligonucleotide feature further comprises second forward and reverse primer pair constructs for amplifying a second target polynucleotide sequence.

15. The method of embodiment 14, wherein the second forward and reverse primer pair constructs are linked by a cleavable linker.

16. The method of embodiment 14 or 15, wherein the first and second forward and reverse primer pair constructs are linked by cleavable linkers.

17. The method of any one of embodiments 14 to 16, wherein the amount of the first forward and reverse primer pair constructs in the feature is less than the amount of the second forward and reverse primer pair constructs in the feature.

18. The method of any one of embodiments 14 to 17, wherein the first and second forward and reverse primer constructs form a nested primer set.

19. The method of any one of embodiments 14 to 17, wherein the first and second forward and reverse primer pair constructs are for amplifying non-overlapping first and second target polynucleotide sequences.

20. The method of any one of embodiments 1 to 19, wherein each of the plurality of oligonucleotide features comprises from 2 to 100 different forward and reverse primer pair constructs.

21. The method of any one of embodiments 1 to 20, wherein each of the plurality of oligonucleotide features comprises the same set of forward and reverse primer pair constructs.

22. The method of any one of embodiments 1 to 20, wherein one or more of the plurality of oligonucleotide features comprises a different set of forward and reverse primer constructs as compared to at least one other oligonucleotide feature.

23. The method of any one of embodiments 1 to 21, wherein one or more of the plurality of features further comprises an immobilized reporter probe, wherein the reporter probe is complementary to a region in the target polynucleotide sequence, the method further comprising performing quantitative PCR.

24. The method of any one of embodiments 1 to 23, wherein the incubating step comprises performing a real time PCR.

25. The method of any one of embodiments 1 to 24, the method further comprising applying a master mix of amplification reagents to each of the plurality of oligonucleotide features.

26. The method of embodiment 25, wherein the applying step is performed before the contacting step.

27. The method of any one of embodiments 1 to 26, wherein the contacting step comprises contacting a set of adjacent features on the array with a single sample.

28. The method of any one of embodiments 1 to 26, wherein one or more of the plurality of features is contacted with a different nucleic acid sample as compared to at least one other feature.

29. The method of any one of embodiments 1 to 28, further comprising immersing the array in an immiscible fluid prior to the incubating step.

30. The method of any one of embodiments 1 to 29, wherein the nucleic acid sample comprises nucleic acids selected from the group consisting of: one or more chromosomes, nucleic acids from a single cell, nucleic acids from a population of cells, nucleic acids from a tissue, and nucleic acids from an organism.

31. An oligonucleotide array for multiplex amplification of target polynucleotides, the array comprising: a plurality of oligonucleotide features, wherein each of the plurality of features comprises substrate-immobilized forward and reverse primer pair constructs comprising target nucleic acid binding sites for amplifying a target polynucleotide sequence.

32. The oligonucleotide array of embodiment 31, wherein the forward and reverse primer pair constructs are immobilized via hybridization of a terminal linker sequence to a complementary sequence on a capture oligonucleotide covalently attached to the substrate.

33. The oligonucleotide array of embodiment 32, wherein the forward and reverse primer pair constructs each comprise different terminal linker sequences.

34. The oligonucleotide array of embodiment 33, wherein the sequence complementary to the terminal linker sequence of the forward primer construct and the sequence complementary to the terminal linker sequence of the reverse primer construct are present on the same capture oligonucleotide.

35. The oligonucleotide array of embodiment 33, wherein the sequence complementary to the terminal linker sequence of the forward primer construct and the sequence complementary to the terminal linker sequence of the reverse primer construct are present on different capture oligonucleotides.

36. The oligonucleotide array of any one of embodiments 33 to 35, wherein the terminal linker sequence of the forward and reverse primer pair constructs are different universal primer sites.

37. The oligonucleotide array of embodiment 31, wherein the forward and reverse primer pair constructs are covalently immobilized to the substrate.

38. The oligonucleotide array of embodiment 37, wherein the forward and reverse primer pair constructs are linked by a cleavable linker to the substrate, wherein the cleavable linker is cleaved prior to or during the incubating step.

39. The oligonucleotide array of any one of embodiments 31, 32, 37 and 38, wherein the forward and reverse primer pair constructs are linked to each other via a cleavable linker, wherein the cleavable linker is cleaved prior to or during the incubating step.

40. The oligonucleotide array of embodiment 38 or 39, wherein the cleavable linker is selected from the group consisting of: a chemically cleavable linker, a photo-cleavable linker, an enzymatically cleavable linker, and combinations thereof.

41. The oligonucleotide array of any one of embodiments 31, 32, 38 and 40, wherein the forward primer construct and/or the reverse primer construct comprises a universal primer site.

42. The oligonucleotide array of any one of embodiments 31 to 41, wherein the forward primer construct and/or the reverse primer construct comprises a barcode sequence upstream of the target nucleic acid binding site and downstream of the universal primer site, when present.

43. The oligonucleotide array of embodiment 42, wherein the barcode sequence is a degenerate barcode sequence.

44. The oligonucleotide array of any one of embodiments 31 to 43, wherein the oligonucleotide feature further comprises second forward and reverse primer pair constructs for amplifying a second target polynucleotide sequence.

45. The oligonucleotide array of embodiment 44, wherein the second forward and reverse primer pair constructs are linked by a cleavable linker.

46. The oligonucleotide array of embodiment 44 or 45, wherein the first and second forward and reverse primer pair constructs are linked by cleavable linkers.

47. The oligonucleotide array of any one of embodiments 44 to 46, wherein the amount of the first forward and reverse primer pair constructs in the feature is less than the amount of the second forward and reverse primer pair constructs in the feature.

48. The oligonucleotide array of any one of embodiments 44 to 47, wherein the first and second forward and reverse primer constructs form a nested primer set.

49. The oligonucleotide array of any one of embodiments 44 to 47, wherein the first and second forward and reverse primer pair constructs are for amplifying non-overlapping first and second target polynucleotide sequences.

50. The oligonucleotide array of any one of embodiments 31 to 49, wherein each of the plurality of oligonucleotide features comprises from 2 to 100 different forward and reverse primer pair constructs.

51. The oligonucleotide array of any one of embodiments 31 to 50, wherein each of the plurality of oligonucleotide features comprises the same set of forward and reverse primer pair constructs.

52. The oligonucleotide array of any one of embodiments 31 to 50, wherein one or more of the plurality of oligonucleotide features comprises a different set of forward and reverse primer constructs as compared to at least one other oligonucleotide feature.

53. A kit for nucleic acid amplification, the kit comprising an oligonucleotide array according to any one of embodiments 31 to 52.

54. The kit of embodiment 53, wherein the kit further comprises a master mix of amplification reagents.

55. The kit of embodiment 53 or 54, wherein the kit further comprises a sequencing primer.

56. The kit of embodiment 55, wherein the kit further comprises sequencing reagents.

57. The kit of any one of embodiments 53 to 57, wherein the kit further comprises instructions for performing the method of any one of embodiments 1 to 30.

58. A method of sequencing, the method comprising: performing a multiplex amplification of target polynucleotides according to any one of embodiments 1 to 30 to produce a pool of amplified target polynucleotides; and sequencing the pool of amplified target polynucleotides.

59. The method of embodiment 58, wherein the sequencing step comprises performing next generation sequencing.

What is claimed is:

1. A method for multiplex amplification of target polynucleotides, the method comprising:
providing a solid substrate comprising a plurality of oligonucleotide features, wherein each of the plurality of oligonucleotide features comprises a substrate-immobilized forward primer construct comprising a first target nucleic acid binding site and a substrate-immobilized reverse primer construct comprising a second target nucleic acid binding site, wherein the first and second target nucleic acid binding sites are for amplifying a target polynucleotide sequence;
contacting each of the plurality of features with a corresponding nucleic acid sample;
incubating the contacted plurality of features under conditions to allow amplification of the target polynucleotide sequence by the forward and reverse primer pair constructs, wherein the forward and reverse primer pair constructs are released from the substrate prior to or during the incubating step.

2. The method of claim 1, wherein the forward and reverse primer pair constructs are immobilized via hybridization of a terminal linker sequence to a complementary sequence on a capture oligonucleotide covalently attached to the substrate.

3. The method of claim 2, wherein the forward and reverse primer pair constructs each comprise different terminal linker sequences.

4. The method of claim 3, wherein the sequence complementary to the terminal linker sequence of the forward primer construct and the sequence complementary to the terminal linker sequence of the reverse primer construct are present on the same capture oligonucleotide.

5. The method of claim 3, wherein the sequence complementary to the terminal linker sequence of the forward primer construct and the sequence complementary to the terminal linker sequence of the reverse primer construct are present on different capture oligonucleotides.

6. The method of claim 2, wherein the terminal linker sequences of the forward and reverse primer pair constructs are universal primer sites.

7. The method of claim 1, wherein the forward and reverse primer pair constructs are covalently immobilized to the substrate.

8. The method of claim 7, wherein the forward and/or reverse primer pair constructs are linked by a cleavable linker to the substrate, wherein the cleavable linker is cleaved prior to or during the incubating step.

9. The method of claim 1, wherein the forward and reverse primer pair constructs are linked to each other via a cleavable linker, wherein the cleavable linker is cleaved prior to or during the incubating step.

10. The method of claim 8, wherein the cleavable linker is selected from the group consisting of: a chemically cleavable linker, a photo-cleavable linker, an enzymatically cleavable linker, and combinations thereof.

11. The method of claim 1, wherein the forward primer construct and/or the reverse primer construct comprises a universal primer site.

12. The method of claim 1, wherein the forward primer construct and/or the reverse primer construct comprises a barcode sequence upstream of the target nucleic acid binding site and downstream of the universal primer site, when present.

13. The method of claim 12, wherein the barcode sequence is a degenerate barcode sequence.

14. The method of claim 1, wherein the oligonucleotide feature further comprises second forward and reverse primer pair constructs for amplifying a second target polynucleotide sequence.

15. The method of claim 14, wherein the second forward and reverse primer pair constructs are linked by a cleavable linker.

16. The method of claim 1, wherein the first and second forward and reverse primer pair constructs are linked by cleavable linkers.

17. The method of claim 14, wherein the amount of the first forward and reverse primer pair constructs in the feature is less than the amount of the second forward and reverse primer pair constructs in the feature.

18. The method of claim 14, wherein the first and second forward and reverse primer constructs form a nested primer set.

19. An oligonucleotide array for multiplex amplification of target polynucleotides, the array comprising:
a plurality of oligonucleotide features, wherein each of the plurality of features comprises substrate-immobilized forward and reverse primer pair constructs comprising target nucleic acid binding sites for amplifying a target polynucleotide sequence.

20. A method of sequencing, the method comprising:
performing a multiplex amplification of target polynucleotides according claim 1 to produce a pool of amplified target polynucleotides; and
sequencing the pool of amplified target polynucleotides.
